(19) United States
(12) Patent Application Publication
(10) Pub. No.: US 2013/0344510 A1
(43) Pub. Date: Dec. 26, 2013

(54) IDENTIFICATION OF CANCER PATIENTS LIKELY TO BENEFIT FROM RADIATION THERAPY

(71) Applicants: Mark Gustavson, Greensboro, NC (US); Jason Christiansen, Glastonbury, CT (US); Anthony Martin Magliocco, Tampa, FL (US)

(72) Inventors: Mark Gustavson, Greensboro, NC (US); Jason Christiansen, Glastonbury, CT (US); Anthony Martin Magliocco, Tampa, FL (US)

(73) Assignees: UTI Limited Partnership, Alberta, CA (US); HistoRx, Inc., Branford, CT (US)

(21) Appl. No.: 13/990,727

(22) PCT Filed: Dec. 2, 2011
(86) PCT No.: PCT/US11/63038

§ 371 Date: Sep. 11, 2013

Related U.S. Application Data
(60) Provisional application No. 61/344,996, filed on Dec. 3, 2010.

(51) Int. Cl. G01N 33/68

(52) U.S. Cl. CPC G01N 33/6893
USPC 435/7.23

(57) ABSTRACT

Disclosed herein are methods of identifying suitable patients for postoperative radiotherapy based on the discovery that the quantification of ER, beyond simple positive/negative characterization, can provide valuable predictive information for the treatment of cancer, specifically breast cancer, and more particularly may predict a group more likely to respond to RT and spare patients from a potentially harmful treatment. Furthermore, the true quantification of ER expression provides a continuous recurrence risk assessment for patients being treated with tamoxifen, and therefore the standardization of the data across sites and imaging platforms significantly reduces the misclassification of patients when compared to the current standard by which ER expression is determined.

### CROSS-REFERENCE TO RELATED PATENT APPLICATIONS

This application claims priority from U.S. Provisional Application No. 61/344,996, filed Dec. 3, 2010, incorporated herein by reference in its entirety.

### FIELD OF THE INVENTION

The present technology relates to methods of identifying cancer patients likely to benefit from radiation therapy and methods of predicting such a patient's response to radiation therapy. In particular, this approach to prescribing radiation therapy involves utilizing standardized quantitative assessments of the level of estrogen receptor expression in a patient's tumor to determine likelihood of success of the therapy.

### BACKGROUND OF THE INVENTION

The following discussion of the background is merely provided to aid the reader in understanding the technology and is not admitted to describe or constitute prior art to the present application.

Radiation therapy (RT) involves the medical use of ionizing radiation as part of a cancer treatment to damage the DNA of malignant cells, either directly or by creating charged particles within the affected cells that damage the DNA. It has applications in curative, adjuvant, and palliative treatment. Over the past several decades, RT has become a step in the standard protocol for treatment of cancers including breast cancer. For early-stage breast cancer patients, the modern standard of care often involves breast-conserving surgery followed by postoperative whole-breast external beam radiotherapy. See Vaidya et al., Targeted intraoperative radiotherapy versus whole breast radiotherapy for breast cancer (TARGIT-A trial): an international, prospective, randomised, non-inferiority phase 3 trial, Lancet, Vol. 376, 91-102, July 2010, which is hereby incorporated by reference in its entirety.

RT itself is painless, and the incidence of side effects is low, however, higher doses can cause both acute and chronic side effects. Which side effects develop depend on the area of the body being treated, the dose given per day, the total dose given, the patient's general medical condition, and other treatments given at the same time, but may include: skin irritation or damage, fatigue, nausea, vomiting, fibrosis, bowel damage, memory loss, infertility, and, rarely, a second cancer. In addition to the physical side effects, the 3-7 week postoperative RT schedule is impracticable for many patients. Therefore, there is a critical need for methods to determine which patients might benefit from RT prior to administration of the therapy, in order to properly balance the potential results of the treatment against the danger and inconvenience of side effects and treatment protocol.

In addition to RT, patients suffering from certain cancers, including breast cancer, may also be treated with hormone therapy. Hormone therapy for breast cancer involves the medical use of hormones to adversely affect the function of the naturally occurring hormones (e.g. estrogen or progesterone) which attach to breast tumor cells and affect their ability to multiply. Generally, it has been demonstrated that hormone therapy involving antiestrogenic agents can be effective if a patient tests positive for estrogen receptors (ERs). However, continuous association between relative risk and quantitative ER expression had not been established. Further, an assessment of whether quantitative levels of ER plays a role in a patient's response to RT was unknown.

### SUMMARY OF THE INVENTION

The present technology is based on the discovery that a patient's response to RT can be predicted by a quantification of ER expression. Provided herein are methods for predicting treatment outcomes using such protein expression analysis and methods of identifying patients who would be likely to benefit from certain courses of treatment.

In one aspect, the present invention provides a method of identifying a cancer patient—in some embodiments, a breast cancer patient—who will likely benefit from radiation therapy comprising: determining a level of estrogen receptor (ER) expression specifically in a population of tumor cells taken from a cancer patient, which determination uses a technique that provides a quantitative analysis of the level of ER expression, determining if a result of the quantitative analysis, manifested by a score taken from a continuous scale, falls above or below a predetermined reference score, and selecting the cancer patient whose score falls below the predetermined reference score as one who will likely benefit from radiation therapy.

In some embodiments, the technique used to provide quantitative analysis includes an objective, reproducible, quantitative, multiparametric analysis of one or more proteins in the tumor sample. Such a technique for determining ER expression may use an automated digital pathology system, or, in further embodiments, a quantitative image analysis procedure. In some embodiments, the predetermined reference score may correspond to a median score expected from a cohort of cancer patients having tumors that are ER positive.

Additionally, in some embodiments, the method of identifying a cancer patient who will likely benefit from RT may further comprise subjecting the cancer patient to hormonal therapy. Such hormonal therapy may be administered before, during, or after radiation therapy. The hormonal therapy may, in some embodiments, include administration of an effective amount of an agent that exhibits nonestrogenic properties. Such an agent may compete with estrogen for binding sites in a tissue, including ER. Further, the agent may include tamoxifen, toremifene, or combinations thereof, steroidal or non-steroidal aromatase inhibitors including anastozole, letrozole, exernestane, or combinations thereof, goserelin, leuprolide, fulvestrant, megastrol acetate, ethinyl estradiol, fluoxymesterone, or combinations thereof.

In another aspect, the present invention provides a method of identifying a cancer patient—in some embodiments, a breast cancer patient—whose tumor is estrogen receptor (ER) positive and who will likely benefit from radiation therapy comprising determining a level of ER expression specifically in a population of tumor cells taken from the patient, which determination uses a technique that provides a quantitative analysis of the level of ER expression on a continuous scale, determining if a result of the quantitative analysis, manifested by a score taken from the continuous scale, falls above or below a predetermined reference score, and selecting the cancer patient whose score falls below the predetermined reference score as one who will likely benefit from radiation therapy.

In yet another aspect, the present invention provides a method of identifying a cancer patient receiving hormonal therapy who will likely benefit from radiation therapy comprising determining a level of ER expression specifically in a population of tumor cells taken from a cancer patient, which determination uses a technique that provides a quantitative analysis of the level of ER expression on a continuous scale, determining if a result of the quantitative analysis, manifested by a score taken from the continuous scale, falls above or below a predetermined reference score, and selecting the cancer patient whose score falls below the predetermined reference score as one who will likely benefit from radiation therapy.

In still another aspect, the present invention provides a kit comprising a first stain specific for a nuclear compartment of a cell, a second stain specific for ER, and instructions for using the contents of the kit, which includes a predetermined reference score against which a result of a quantitative analysis of a level of ER expression within the nuclear compartment of a cell can be compared. In some embodiments, the kit may further contain a third stain specific for epithelial cytoplasmic subcellular compartment.

In a final aspect, the present invention provides a method of identifying a cancer patient who will likely benefit from radiation therapy comprising determining a level of ER expression specifically in a population of tumor cells taken from a cancer patient, which determination uses a computer-assisted method for quantifying ER expression within a subcellular compartment of individual cells of said population of tumor cells, which method includes: incubating the tumor sample with a first stain that specifically labels a nuclear compartment of individual cells and a second stain that specifically labels ER, obtaining a high resolution image of each of the first and the second stains retained in the tissue sample using an automated digital pathology system to provide a first image of the nuclear compartment and a second image of ER, analyzing the first image to identify image pixels that represent the nuclear compartment, analyzing the second image to identify image pixels that represent ER and determining a total intensity value of the image pixels that represent ER and which reside within the nuclear compartment, rendering a score for the cancer patient by dividing the total intensity value by a total area of the nuclear compartment, and selecting the cancer patient whose score falls below a predetermined reference score as one who will likely benefit from radiation therapy. In some embodiments, the predetermined reference score may fall between about 9000 and about 9025.

As used herein, “AQUA” technology refers to a fluorescence-based platform enabling objective and completely standardized quantification of protein expression in tissue with minimized operator interaction that provides tumor-specific, quantitative and continuous expression score data. AQUA technology is described in detail in U.S. Pat. No. 7,219,016 and published US Patent Application Nos. US2009/0034823 and US2010/136549 the disclosures of which are hereby incorporated in their entirety by reference herein.

As used herein, “patient” refers to a human that has been diagnosed with a cancer generally treated with radiation therapy or as having an increased likelihood of developing a cancer generally treated with radiation therapy. A patient may also be subjected to other treatment regimens, in addition to radiation therapy, including but not limited to surgery, chemotherapy, hormonal or endocrine therapy, or combinations thereof.

As used herein, “effective amount” of a drug or agent is an amount of a drug that, when administered to a patient with cancer, will have the intended therapeutic effect, e.g., alleviation, amelioration, palliation or elimination of one or more manifestations of cancer in the patient. The full therapeutic effect does not necessarily occur by administration of one dose (or dosage), and may occur only after administration of a series of doses. Thus, a therapeutically effective amount may be administered in one or more administrations.

As used herein, the terms “antiestrogenic agent” or an agent that exhibits “nonestrogenic properties” are used interchangeably to describe an agent that inhibits the usual production or function of estrogen in the body. For example, such antiestrogenic agents may competitively bind to target tissue to exclude estrogen, or may decrease estrogen production by inhibiting output of pituitary gonadotropins.

As used herein, “estrogen receptor expression” or “ER expression” refers to the presence of the nuclear hormone intracellular receptor for estrogen, not including the estrogen G-protein-coupled receptor. ER expression may also refer to a level of receptor presence within a cell or a subcellular compartment.

As used herein, “determining a level of ER expression” refers to establishing via biomolecular methods the amount or concentration of ERs expressed by the ESTI gene in a given tumor sample, generally by employing a quantitative analysis.

As used herein, a “quantitative analysis” refers to a biomolecular determination of the absolute or relative abundance of a particular substance, e.g., ER, present in a sample. In particular, a quantitative analysis of the level of ER expression refers herein to a determination of the absolute number or concentration of ERs in a given tumor sample. A quantitative analysis may be conducted for a given protein using variations on immunohistochemistry techniques, or using other methods as described herein.

As used herein, a “cohort” refers to a group of subjects or specimens which share certain defining characteristics, for example, tissue type (e.g., tumor sample), tamoxifen treatment status, and/or ER-positive status.

As used herein, a “computer-assisted method” refers to any immunohistochemistry analysis with at least one step, e.g., steps comprising quantitative analysis, performed or regulated by a computer or other programmable machine that inputs, stores, or manipulates data, or provides output in a useful format.

As used herein, “tumor sample” refers to an invasive breast cancer specimen from a patient which consists essentially of a population of tumor cells. In a preferred embodiment, ER expression in the tumor sample is specifically quantified in tumor cells (i.e. AQUA analysis) or, in another preferred embodiment, the tumor sample is substantially free of stroma and other undesirable components that may be present in surrounding connective tissue and which may confound a quantitative protein analysis. That is, a tumor sample should be obtained and prepared or otherwise examined such that a quantitative analysis of the level of ER expression can be attributed substantially, if not solely, to ER expression in tumor cells.

As used herein, a “predetermined reference score” refers to a standardized quantitative value reflecting biomarker expression, in this case ER expression according to a specified methodology that differentiates patient outcome such as response to a specific treatment. For example the predetermined reference score may be a cut point such that patients having a biomarker score above the cut point are not expected to respond to a specific treatment, whereas patients having a biomarker score below the cut point are expected to respond to the specific treatment. The predetermined reference score may be the median value of the score distribution frequency curve of a cohort of patients.

### BRIEF DESCRIPTION OF THE FIGURES

FIG. 1 illustrates two examples of in situ protein expression measurement for ER in tumor samples using a continuous quantitative assessment performed using AQUA technology to obtain a standardized score for ER. One example illustrates a high ER AQUA score, the other a low ER AQUA score.

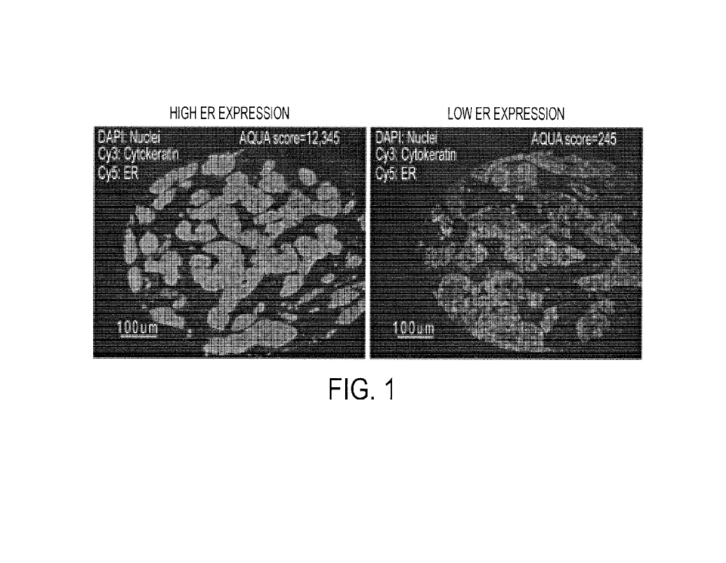

FIGS. 2(A) and (B) illustrate the site-to-site standardization achieved by the ER AQUA assay. Linear regression analysis appears in (A), and an overall AQUA score means comparison appears in (B), for two independent sites.

FIGS. 3(A) and (B) illustrate the reproducibility of the scores achieved by the ER AQUA assay. The same set of Tissue Microarray (TMA) slides was run on two independent imaging platforms, with resulting linear regression data shown in (A), and an overall AQUA score means comparison shown in (B).

FIGS. 4(A) and (B) show the distribution and mean of ER AQUA scores of one cohort of breast cancer patients, and a comparison of the ER expression of those patients with cancer recurrence with the ER expression of those patients with no cancer recurrence.

FIG. 5 demonstrates the greater benefits from RT accruing to patients with ER AQUA scores below the median AQUA score (left) when compared to patients with ER AQUA scores above the median AQUA score (right).

### DETAILED DESCRIPTION

The present technology relates generally to methods for identifying cancer patients likely to benefit from radiation therapy and methods of predicting such a patient's response to radiation therapy.

In a first aspect, the methods include a step of determining a level of estrogen receptor (ER) expression specifically in a population of tumor cells taken from a cancer patient, which determination uses a technique that provides a quantitative analysis of the level of ER expression.

### ER Expression

Estrogen receptors, in particular ERα, are found in many cells, but notably in endometrium, breast tumor cells, ovarian stroma cells, and the hypothalamus. The main function of ER is as a DNA binding transcription factor that regulates gene expression, which is generally activated by 17β-estradiol (estrogen). ERα is coded for by the ESR1 gene.

Estrogen is implicated in the development of cancers such as breast cancer, based on data from both clinical and animal studies; risk factors associated with breast cancer reflect cumulative exposure of the breast epithelium to estrogen. Two current hypotheses exist to explain this relationship. In the first, binding of estrogens to the ER stimulates proliferation of breast cells, increasing the target cell number within the tissue, and the increase in cell division and DNA synthesis elevates the risk for replication errors, which may result in the acquisition of detrimental mutations that disrupt normal cellular processes such as apoptosis, cellular proliferation, or DNA repair. In the second hypothesis, estrogen metabolism leads to the production of genotoxic by-products that could directly damage DNA, again resulting in point mutations. There is evidence that estrogen may act through both mechanisms to initiate and/or promote breast cancer. For a complete discussion, see Deroo and Korach, Estrogen receptors and human disease, J. Clin. Invest., 116(3): 561-570, 2006, which is hereby incorporated by reference in its entirety.

ER function may be affected by selective estrogen receptor modulators (SERMs), such as tamoxifen or toremifene, which have been considered an effective adjuvant hormone therapy for breast cancer for several decades. Tamoxifen acts as an estrogen antagonist by binding to helix 12 of ERα and recruiting a corepressor complex such as SMRT or NCoR to estrogen-regulated genes in breast tumor or cancer cells. ER function can further be affected by steroidal or non-steroidal aromatase inhibitors such as, for example, anastozole, letrozole, or exemestane, or combinations thereof. Aromatase inhibitors act to inhibit production of the enzyme aromatase, which converts the hormone androgen into estrogen, thereby decreasing the amount of available estrogen for ER binding. Finally, ER function may be affected by a host of other agents used in hormone therapy, such as goserelin, leuprolide, fulvestrant, megestrol acetate, fluoxymesterone, or combinations thereof. These agents act as gonadotropin releasing hormone agonists or estrogen receptor antagonists.

However, many of the abovementioned hormone therapies are dependent upon the presence of ER in the tumor cells. Indeed, since the ER-dependent hormone therapies are mostly ER antagonists, they are generally only effective if the tumor cell expresses ER. The results of the analysis of ER expression in common use currently reports whether the tumore is ER negative or ER positive. It is standard of care for ER positive patients to be treated with hormonal therapy. Since the currently used test for ER reveals only the patient's status as ER-positive or ER-negative and does not give a quantitative score on a continuous scale of ER expression, it can not further differentiate those patients that may benefit from RT therapy and most ER-positive patients are currently given RT in addition to hormone therapy.

### Quantitative Determination

The present invention provides methods of determining which patients might benefit from RT, the first steps of which require a quantitative determination of the level of ER expression. This determination may be effected using any technique capable of measuring a quantitative level of protein expression, but preferably a technique that is an objective, reproducible, quantitative, multiparametric analysis of one or more proteins in a given tumor sample.

A tumor sample for use in the present invention should be taken as an invasive tissue sample (e.g., a needle biopsy). While material other than tumor cells such as stroma or other neighboring tissue may be present in the population of tumor cells, it is important that the analysis of ER expression is specifically quantified in the tumor cells. If analysis is conducted on a cell lysate, it is important that tissue other than tumor cells be removed prior to analysis.

As described by Cregger et al., Immunohistochemistry and Quantitative Analysis of Protein Expression, Arch. Pathol. Lab. Med., Vol. 130, 2006, which is hereby incorporated by reference in its entirety, the traditional means of quantitatively assessing protein expression is through immunohistochemistry (IHC) techniques. Immunohistochemistry involves a series of uniform steps, typically beginning with antigen retrieval. Methods of antigen retrieval vary in terms of reagents and methods. The process may involve pressure cooking, protease treatment, microwaving, or heating histologic sections in baths of appropriate buffers, with the standard goal of unmasking antigens hidden by formalin cross-links or other fixation. The first definitive step of IHC following antigen retrieval is the application of a specific primary antibody (typically produced by immunizing a mouse or rabbit with a peptide/antigen of interest), followed by extensive washing to remove excess amounts of primary antibody. A species-specific secondary antibody is then applied, which binds to the primary antibody. The secondary antibody is typically conjugated to biotin, horseradish peroxidase, or some other tag. Finally, a detection reagent is applied that includes a chromagen or a fluorescently tagged molecule to visualize the localization of the primary antibody. Traditional IHC, however, has inherent variability. Sources of variability include fixation conditions, specimen pretreatment, reagents, detection methods, and interpretation of results.

Certain platforms have been developed in an attempt to overcome the limitations of IHC. Automated pathology systems, which are systems that mechanize and computerize the IHC process using tissue microarrays (TMAs) and automated staining and slide preparation in order to eliminate variability to the degree possible, have evolved in response to the limitations of traditional IHC. TMAs provide an important step in automation, allowing all tissue samples to be exposed to identical conditions. Computer assistance is also necessary to eliminate intraobserver and interobserver variability. Such automated systems may include automated staining (conventional stains, histochemical techniques, immunostainers); automated in situ hybridization systems; automatic slide preparation (coverslip, slide drying) and integrated slide and cassette labeling, as described in Roja et al., Review of imaging solutions for integrated, quantitative immunohistochemistry in the Pathology daily practice, Folia Histochemica et Cytobiologica, Vol. 47, No. 3, 349-354, 2009, which is hereby incorporated by reference in its entirety.

Further, quantitative image analysis procedures have been developed in response to demand for a biologically accurate system of analysis, which is not provided by a simple result of positive or negative for the desired protein. Quantitative image analysis procedures are those which perform quantitative protein analysis using images obtained with the help of virtual or digital microscopy and whole-slide imaging. Stains are often used in image analysis to allow visualization of target material. Apart from staining, such procedures require an optical microscope system, an image acquisition system, software that controls the scan process, a digital slide viewer, and, optionally, an image processing system and/or a slide feeder. For a more complete description, see Rojo et al, Critical Comparison of 31 Commercially Available Digital Slide Systems in Pathology, International Journal of Surgigal Pathology, 2006, 14:285.

For use in the present methods, a high-resolution image of the nuclear compartment and ER is desired. In order to obtain a high-resolution image in the presently claimed methods, a camera or image generator capable of 1024/1024 pixels or greater should preferably be utilized.

Assays for molecular quantification that do not require masceration of the tissues and cells and the loss of spatial information that necessitates are rare. However, there are a number of computer-based programs designed specifically for the quantitative analysis of IHC. Systems for which publications can be found include BLISS and IHCscore of Bacus Laboratories, Inc (Lombard, Ill.); ACIS of Clarient, Inc (San Juan Capistrano, Calif.); iVision and GenoMx of BioGenex (San Ramon, Calif.); ScanScope of Aperio Technologies (Vista, Calif.); Ariol SL-50 of Applied Imaging Corporation (San Jose, Calif.); LSC Laser Scanning Cytometer of CompuCyte Corporation (Cambridge, Mass.); and AQUA of HistoRx Inc (New Haven, Conn.). Cregger et al., 2006. These platforms generally include computer-assisted digital microscopes with the ability to count cells that have been stained or marked in some way (e.g., fluorescence).

Unlike traditional fluorescence IHC, technology in a system such as the AQUA system is objective and produces strictly quantitative in situ protein expression data on a continuous scale. The AQUA system takes advantage of the multiplexing power of fluorescence by using multiple markers to molecularly differentiate cellular and sub-cellular compartments within which simultaneous quantification of biomarkers-of-interest can be performed. In addition, AQUA analysis provides for standardization and a high degree of reproducibility with % CVs less than 5%, which is superior to any chromagen-based IHC quantification system available to date.

Using AQUA technology, a standardized score is produced which quantifies the level of protein expression. The system and/or software are configured to process the acquired digital images to automatically detect, localize, and quantitate measurement of protein biomarker intensity. The AQUA score is automatically determined from one or more digital images of the tissue sample, the scoring being reflective of biomarker expression level in a defined compartment in the tissue section.

AQUA technology employs a computer-assisted method for quantifying ER expression within a subcellular compartment of individual cells of said population of tumor cells. The tumor sample is first incubated with a first stain that specifically labels a nuclear compartment of individual cells and a second stain that specifically labels ER, obtaining a high resolution image of each of the first and the second stains retained in the tissue sample using an automated digital pathology system to provide a first image of the nuclear compartment and a second image of ER. The first image is then analyzed to identify image pixels that represent the nuclear compartment, and the second image is analyzed to identify image pixels that represent ER. The total intensity value of the image pixels that represent ER and which reside within the nuclear compartment is then determined, and a score for the cancer patient is rendered by dividing the total intensity value by a total area of the nuclear compartment.

### Quantitative Score Comparison

The methods of the present invention further comprise steps of determining if the result of the quantitative analysis, manifested by a score taken from a continuous scale, falls above or below predetermined reference score garnered from ER positive tumors of a cohort of cancer patients.

A predetermined IHC or AQUA reference score, or a predetermined reference value of any other quantitative score set determined using automated digital pathology or quantitative image analysis procedure, may be chosen by performing a score distribution frequency analysis according to methods well-known in the art. For the presently-claimed methods, the predetermined reference score must be determined using a score distribution frequency analysis of scores taken from a continuous scale, i.e. not dichotomous, in which any numerical value greater than or equal to zero may be achievable. If the predetermined reference value is the median value of the score distribution frequency curve, which is the value that divides a frequency distribution into two equal parts, it may be calculated by equations (I) or (II), assuming that X₁, X₂, X₃, . . . Xₙ is a set of data arranged in ascending order of magnitude:

(I) Mₑ=X_{(n+1)/2}, if n is odd,

(II) Mₑ=(X_{n/2}+X_{(n/2+1)}), if n is even.

Once established for a given technique, a predetermined reference score, for example a median AQUA score, can be used for comparison with any patient's level of ER expression determined using the same platform. In consultation with physicians, additional factors may be weighted in determining the reference value for each platform.

In preferred embodiments, since the assessment of ER expression is objective, reproducible, quantitative, and multiparametric, a patient's score from such an assessment may be directly compared to a predetermined reference score to determine whether it is higher or lower than the reference score.

### RT Expected Benefits

The methods of the present invention further comprise a step of selecting the cancer patient whose score falls below the predetermined reference score as one who will likely benefit from radiation therapy.

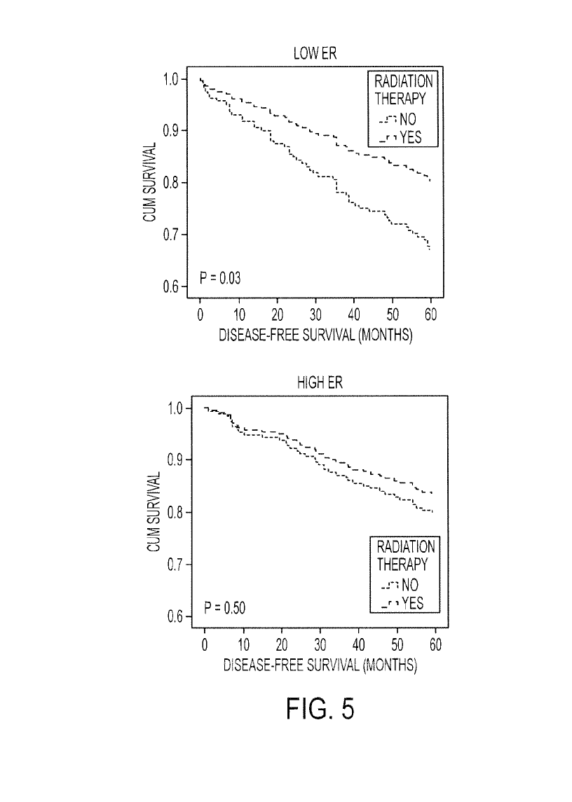

Mean ER scores have been shown herein to be significantly higher in patients who have not had disease recurrence at five years when compared to disease recurrent patients at five years, which suggests that increased ER expression has a protective effect with respect to recurrence. However, it is also demonstrated herein (see, e.g., FIG. 5) that only patients with ER expression levels falling below the median benefit from RT. In the example presented here, the median of the measured expression of ER is used to split patient data into high and low expressing groups. However, a clinically viable cutpoint may also be selected based on rational statistical modeling which can dichotomize the data in such a way as the effect is manifested with optimal specificity and sensitivity. Low-level ER expressing patients show significant benefit from RT, whereas RT showed no benefit in high level ER expressing patients. Therefore, the response to RT can be predicted by comparing the quantification of the patient's ER expression to a predetermined reference score of ER expression. For determining optimal treatment for an individual patient, the physician may take into consideration the patient's quantitative ER expression score and therefore their likelihood of benefiting from RT and factor it into other patient information to guide the selection of treatment.

The inventors have discovered that the quantification of ER, beyond simple positive/negative characterization, can provide valuable predictive information for the treatment of cancer, specifically breast cancer, and more particularly may predict a group more likely to respond to RT and spare patients from a potentially harmful treatment. Furthermore, the inventors have determined that true quantification of ER expression provides a continuous recurrence risk assessment for patients being treated with tamoxifen, and therefore the standardization of the data across sites and imaging platforms significantly reduces the misclassification of patients when compared to the current standard by which ER expression is determined.

### Kits

Additional aspects of the present invention provide for kits to be used in patient treatment comprising a first stain specific for a nuclear compartment of a cell, a second stain specific for ER, and instructions for using the contents of the kit, which includes a predetermined reference score against which a result of a quantitative analysis of a level of ER expression within the nuclear compartment of a cell can be compared. The stains may be applied to a tumor sample as part of a computer-assisted platform or any other platform generating a quantitative, continuous ER expression value.

Suitable stains for use in the kits of the present invention include but are not limited to: Anti estrogen receptor antibodies such as 1D5, PharmDx (1D5 and ER-2-123). 6F11, ER88, SP1, Fluorophores that may be conjugated to a primary antibody include but are not limited to Fluorescein, Rhodamine, Texas Red, Cy2, Cy3, Cy5, VECTOR Red, ELF™ (Enzyme-Labeled Fluorescence), Cy0, Cy0.5, Cy1, Cy1.5, Cy3, Cy3.5, Cy5, Cy7, Fluor X, Calcein, Calcein-AM, CRYPTOFLUOR™'S, Orange (42 kDa), Tangerine (35 kDa), Gold (31 kDa), Red (42 kDa), Crimson (40 kDa), BHMP, BHDMAP, Br-Oregon, Lucifer Yellow, Alexa dye family, N-[6-(7-nitrobenz-2-oxa-1,3-diazol-4-yl)amino]caproyl] (NBD), BODIPY™, boron dipyrromethene difluoride, Oregon Green, MITOTRACKER™ Red, DiOC.sub.7 (3), DiIC.sub.18, Phycoerythrin, Phycobiliproteins BPE (240 kDa) RPE (240 kDa) CPC (264 kDa) APC (104 kDa), Spectrum Blue, Spectrum Aqua, Spectrum Green, Spectrum Gold, Spectrum Orange, Spectrum Red, NADH, NADPH, FAD, Infra-Red (IR) Dyes, Cyclic GDP-Ribose (cGDPR), Calcofluor White, Lissamine, Umbelliferone, Tyrosine and Tryptophan. A wide variety of other fluorescent probes are available commercially.

Further amplification of the signal can be achieved by using combinations of specific binding members, such as antibodies and anti-antibodies, where the anti-antibodies bind to a conserved region of the target antibody probe, particularly where the antibodies are from different species. Alternatively specific binding ligand-receptor pairs, such as biotin-streptavidin, may be used, where the primary antibody is conjugated to one member of the pair and the other member is labeled with a detectable probe. Thus, one effectively builds a sandwich of binding members, where the first binding member binds to the cellular component and serves to provide for secondary binding, where the secondary binding member may or may not include a label, which may further provide for tertiary binding where the tertiary binding member will provide a label.

The secondary antibody may be labeled using avidin, strepavidin or biotin, which are each independently labeled with a detectable moiety, such as a fluorescent dye (stain), a luminescent dye or a non-fluorescent dye. In principle, an enzyme that (i) can be conjugated to or bind indirectly to (e.g., via conjugated avidin, strepavidin, biotin, secondary antibody) a primary antibody, could be used. The enzyme employed can be, for example, alkaline phosphatase, horseradish peroxidase, beta-gal actosidase and/or glucose oxidase. The enzyme can also be directed at catalyzing a luminescence reaction of a substrate, such as, but not limited to, luciferase and aequorin, having a substantially non-soluble reaction product capable of luminescencing or of directing a second reaction of a second substrate, such as but not limited to, luciferine and ATP or coelenterazine and Ca.sup.++, having a luminescencing product.

The present technology, thus generally described, will be understood more readily by reference to the following examples, which are provided by way of illustration and are not intended to be limiting.

### EXAMPLES

### Example 1

### Sample Selection, Demographic Summary and Survival Summary

A cohort (n=568) of retrospectively collected invasive breast cancer specimens in triplicate (Tissue Microarray format) from patients treated with 20 mg tamoxifen and with and without RT was assembled. The samples included negative and positive nodal status and were collected from tumors ranging in size from less than 2 cm to greater than 5 cm. Median disease-specific survival for the cohort is 118 months. Table 1 provides the results of Univariate Cox Proportional Hazards (15-year disease specific survival) model examining the effects of the major clinical variables found in the cohort: age, nodal status, and tumor size. Data provided include number and percentage of the cohort in each category, hazards ratio (HR), 95% confidence intervals (95% Cl), and P-values.

TABLE 1

| Parameter | n (%)* | HR | 95% CI | P-value\hat{ } |
|---|---|---|---|---|
| Age | 568 (100) | 1.07 | 1.05-1.09 | <0.001 |
| Nodal Status | | | | |
| Negative | 361 (74) | | | |
| Positive | 127 (26) | 5.30 | 3.50-8.03 | <0.001 |
| Tumor Size | | | | |
| <2 cm | 348 (67) | | | |
| 2-5 cm | 160 (31) | 1.74 | 1.18-2.57 | 0.006 |
| >5 cm | 14 (2) | 5.26 | 2.51-11.04 | <0.001 |
| Radiation Treatment | | | | |
| No | 202 (37) | | | |
| Yes | 350 (63) | 0.85 | 0.58-1.23 | 0.384 |

### Example 2

### Quantitative Assessment of ER Expression of Undivided Cohort

Fluorescence immunohistochemistry with AQUA technology was used to quantitatively assess ER expression (clone 1D5) on the cohort of Example 1. Staining, image acquisition and AQUA analysis was performed at two independent sites using two different standardized digital pathology platforms.

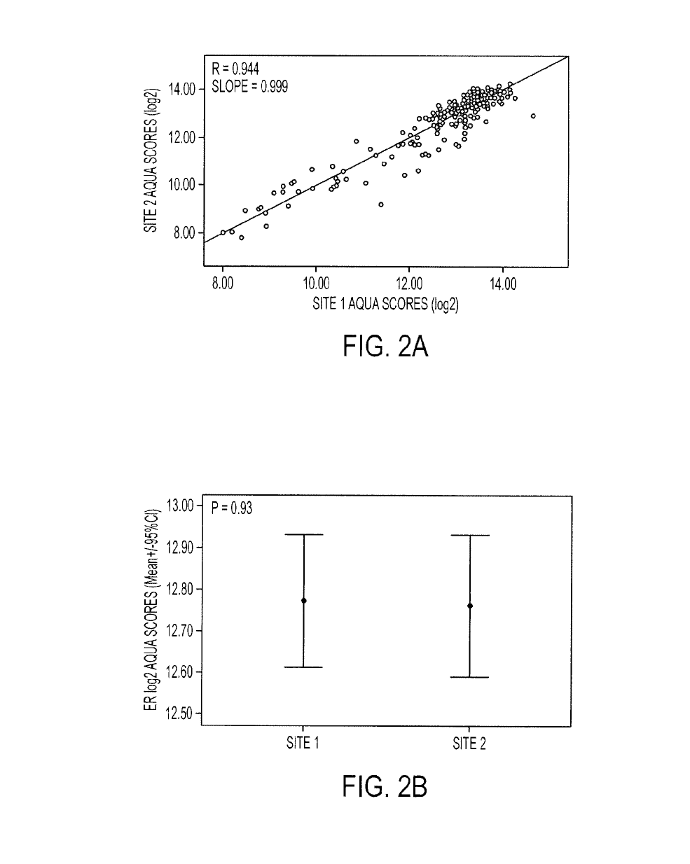

A comparison between completely independent sample staining and quantitative assessment at the two sites showed highly significant correlation, with AQUA scores approaching unity (Pearson's R=0.94; linear slope=0.999) and indistinguishable means (p=0.93). FIG. 2(A) demonstrates the results of an ER AQUA assay, image acquisition and analysis run in parallel at two independent sites on a subset of the cohort (n=245 cases). Linear regression analysis demonstrated a high degree of precision. Further, in FIG. 2(B), an overall AQUA score means comparison demonstrated AQUA scores to be indistinguishable between the two independently run assays.

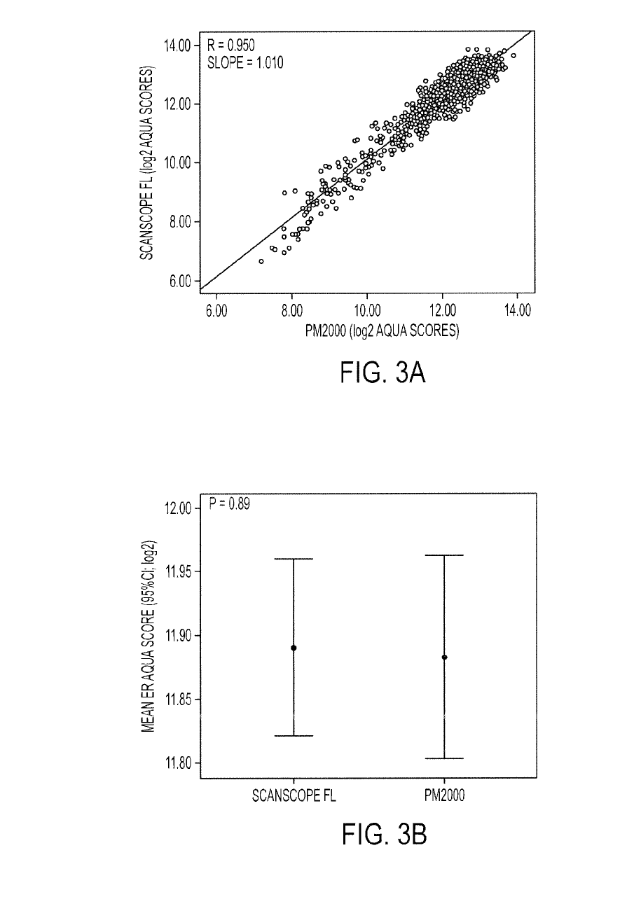

As demonstrated in FIG. 3(A), assessment of the same tissue slides on two unique independent instrument platforms (HistoRx PM2000 v. Aperio Scanscope FL) also showed highly significant correlation. Linear regression analysis on a spot-by-spot basis demonstrated a high degree of precision between imaging platforms (Pearson's R=0.95; linear slope=1.01; p=0.89). Overall AQUA score means comparison in FIG. 3(B) confirmed the AQUA scores to be indistinguishable between the two imaging platforms.

Finally, continuous ER AQUA scores showed a highly significant association with 5-year disease-free survival both by itself (HR=0.80; 95% Cl=0.70-0.91; p=0.001) and when put into a model with nodal status and tumor size (HR=0.60; 95% Cl=0.68-0.94; p=0.006). This data, as shown in Table 2, shows that ER has a highly significant association with survival in all models. These data further indicate that true quantification of ER expression provides a continuous recurrence risk assessment for patients being treated with tamoxifen.

TABLE 2

| | | | | Adjusted for Nodal Status | | |
|---|---|---|---|---|---|---|
| | Univariate | | | and Tumor Size | | |
| | HR | 95% CI | P-value | HR | 95% CI | P-value |
| 5-year DFS | 0.80 | 0.70-0.91 | 0.001 | 0.80 | 0.68-0.94 | 0.006 |
| 15-year OS | 0.83 | 0.72-0.96 | 0.010 | 0.79 | 0.67-0.93 | 0.005 |

### Example 3

### Assessment of Cohort Divided at Quantitative Median AQUA Score

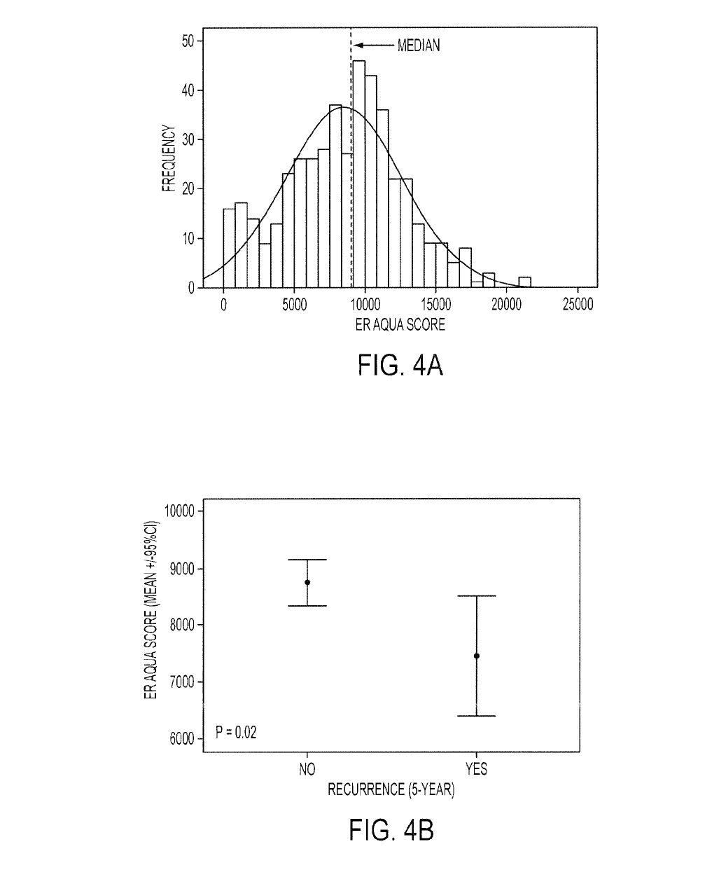

The cohort of Example 2 was then divided at the median AQUA score representing relative low and high ER expressing patients, as shown in FIGS. 4(A) and (B). The ER AQUA scores showed a near-normal distribution in the tamoxifen-treated cohort. The low ER expressing group, which would ordinarily obtain a smaller benefit from treatment with tamoxifen and therefore have a higher recurrence rate, as shown in FIG. 4(B), showed significant benefit from RT for 5-year disease-free survival (HR=0.56; 95% Cl=0.32-0.95; p=0.03) and maintained significance at the 10% level when the model was adjusted for nodal status and tumor size (HR=0.60; 95% Cl=0.32-1.10; p=0.097). In contrast, the high ER expressing group showed no benefit for radiation treatment (HR=0.81; 95% Cl=0.43-1.52; p=0.51).

These results are illustrated in FIG. 5, in which the tamoxifen-treated patient cohort is dichotomized on the median AQUA score (9000) to split the population into low (left) and high (right) ER expressing patients. Cox Proportional Hazards model was performed for each group to examine the effects of radiation treatment on survival. Survival curves are shown for univariate analysis for low and high ER expressing patient groups.

### EQUIVALENTS

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure. Applicants reserve the right to physically incorporate into this application any and all materials and information from any such articles, patents, patent applications, or other physical and electronic documents.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, includes the number recited and refers to ranges which can be subsequently broken down into subranges as discussed above. Finally, as will be understood by one skilled in the art, a range includes each individual member.

Other embodiments are set forth in the following claims.

What is claimed is:

1. A method of identifying a cancer patient who will likely benefit from radiation therapy comprising:
determining a level of estrogen receptor (ER) expression specifically in a population of tumor cells taken from a cancer patient, which determination uses a technique that provides a quantitative analysis of the level of ER expression;
determining if a result of the quantitative analysis, manifested by a score taken from a continuous scale, falls above or below a predetermined reference score; and
selecting the cancer patient whose score falls below the predetermined reference score as one who will likely benefit from radiation therapy.

2. The method of claim 1 in which the cancer patient has been diagnosed as having breast cancer.

3. The method of claim 1 in which the predetermined reference score corresponds to a median score expected from a cohort of cancer patients having tumors that are ER positive.

4. The method of claim 1 in which the level of ER expression is determined using an automated digital pathology system.

5. The method of claim 1 in which the level of ER expression is determined using a quantitative image analysis procedure.

6. The method of claim 1 in which the cancer patient is treated with hormonal therapy in addition to radiation therapy.

7. The method of claim 6 in which the hormonal therapy is administered before, during, or after radiation therapy.

8. The method of claim 6 in which the hormonal therapy includes administering an effective amount of an agent that exhibits nonestrogenic properties.

9. The method of claim 8 in which the agent can compete with estrogen for binding sites in a tissue, including ER.

10. The method of claim 6 in which the hormonal therapy includes administering an effective amount of an agent that exhibits aromatase inhibitory properties.

11. The method of claim 10 in which the agent includes steroidal or non-steroidal aromatase inhibitors.

12. The method of claim 11 in which the agent includes anastozole, letrozole, exernestane, or combinations thereof.

13. The method of claim 6 in which the hormonal therapy includes administering an effective amount of an agent that includes goserelin, leuprolide, fulvestrant, megastrol acetate, ethinyl estradiol, fluoxymesterone, or combinations thereof.

14. The method of claim 1 in which the technique includes an objective, reproducible, quantitative, multiparametric analysis of one or more proteins in the tumor sample.

15. A method of identifying a cancer patient whose tumor is estrogen receptor (ER) positive and who will likely benefit from radiation therapy comprising:
determining a level of ER expression specifically in a population of tumor cells taken from the patient, which determination uses a technique that provides a quantitative analysis of the level of ER expression on a continuous scale;
determining if a result of the quantitative analysis, manifested by a score taken from the continuous scale, falls above or below a predetermined reference score, and
selecting the cancer patient whose score falls below the predetermined reference score as one who will likely benefit from radiation therapy.

16. The method of claim 15 in which the cancer patient has been diagnosed as having breast cancer.

17. A method of identifying a cancer patient receiving hormonal therapy who will likely benefit from radiation therapy comprising:
determining a level of estrogen receptor (ER) expression specifically in a population of tumor cells taken from a cancer patient, which determination uses a technique that provides a quantitative analysis of the level of ER expression on a continuous scale;
determining if a result of the quantitative analysis, manifested by a score taken from the continuous scale, falls above or below a predetermined reference score, and
selecting the cancer patient whose score falls below the predetermined reference score as one who will likely benefit from radiation therapy.

18. A kit comprising:
a first stain specific for a nuclear compartment of a cell;
a second stain specific for estrogen receptor (ER); and
instructions for using the contents of the kit, which includes a predetermined reference score against which a result of a quantitative analysis of a level of ER expression within the nuclear compartment of a cell can be compared.

19. The kit of claim 18 which further comprises a third stain specific for epithelial cytoplasmic subcellular compartment.

20. A method of identifying a cancer patient who will likely benefit from radiation therapy comprising:
determining a level of estrogen receptor (ER) expression specifically in a population of tumor cells taken from a cancer patient, which determination uses a computer-assisted method for quantifying ER expression within a subcellular compartment of individual cells of said population of tumor cells, which method includes:
incubating the tumor sample with a first stain that specifically labels a nuclear compartment of individual cells and a second stain that specifically labels ER;
obtaining a high resolution image of each of the first and the second stains retained in the tissue sample using an automated digital pathology system to provide a first image of the nuclear compartment and a second image of ER;
analyzing the first image to identify image pixels that represent the nuclear compartment;
analyzing the second image to identify image pixels that represent ER and determining a total intensity value of the image pixels that represent ER and which reside within the nuclear compartment;
rendering a score for the cancer patient by dividing the total intensity value by a total area of the nuclear compartment;
selecting the cancer patient whose score falls below a predetermined reference score as one who will likely benefit from radiation therapy.

21. The method of claim 20 in which the predetermined reference score falls in the range of about 9000 to about 9025.
